(19) United States
(12) Patent Application Publication
(10) Pub. No.: US 2024/0288716 A1
(43) Pub. Date: Aug. 29, 2024

(54) VIRTUAL REALITY HEAD-MOUNTED DISPLAY DEVICE WITH BUILT-IN STRABISMUS TREATMENT AND OPERATION METHOD THEREOF

(71) Applicant: FENG CHIA UNIVERSITY, Taichung City (TW)

(72) Inventors: MING-HAN TSAI, Taipei City (TW); JUI-PANG LU, Taoyuan City (TW); HUI-JU LIN, Taichung City (TW)

(21) Appl. No.: 18/141,361

(22) Filed: Apr. 28, 2023

(30) Foreign Application Priority Data
Feb. 25, 2023 (TW) 112107179

(51) Int. Cl. G02C 7/14; A61B 3/08; G02B 27/00; G02B 27/01; G06F 3/01

(52) U.S. Cl. CPC G02C 7/14; A61B 3/085; G02B 27/0093; G02B 27/0101; G02B 27/017; G06F 3/013; G02B 2027/0138; G02B 2027/014

(57) ABSTRACT

The invention is a virtual reality head-mounted display device with built-in strabismus treatment and operation method thereof. The head-mounted display device detects the motion of the user's eyes to get a compensated angle for strabismus. The processing module can generate a compensated image by a compensated angle for the user. While virtual reality devices are operating, our proposed head-mounted display device automatically detects the movement of the user's eyes and increases or decreases the correction effect of the simulated prism diopter lens. The user uses the device for a long time that can reduce the user's strabismus degree and achieve visual correction and treatment.

### TECHNICAL FIELD

The present invention provides a virtual reality head-mounted display device with built-in strabismus treatment, especially a head-mounted device integrate prism diopter simulation system.

### BACKGROUND OF RELATED ARTS

Human beings can see the outer real view via eyes, but the eye is the same as other organs, people will get vision disorders by abnormal eyes caused by genetics or environmental reason. The refractive error is out of focus (such as shortsightedness and longsightedness) or misaligned (such as strabismus or astigmatism).

Both eyes of a healthy individual are aligned and the visual axes are parallel under most viewing conditions. Strabismus is a visual disorder in which the eyes are misaligned. The primary therapeutic goal for strabismus is a comfortable, single, clear, normal binocular vision at all distances and directions of gaze. Strabismus is usually treated with a combination of eyeglasses and surgery.

The strabismus test includes mainly Hirschberg test, cover-uncover test, prism cover test. The Hirschberg test is the inspector focus on a fix object with a certain distance. In a person with normal ocular alignment the light reflex lies slightly nasal from the center of the cornea (i.e. the light reflex is symmetrical). Otherwise, a person with strabismus the light reflex is unsymmetrical. The deviation angle can be measured by a suitable prism. The second test is a cover-uncover test. The patient focus on an object with a short distance or a long distance and a cover is placed over an eye. The patient is required to fixate on an object while the examiner repeats the test several times using prism lenses with a variety of strengths and directions in order to measure the deviation angles. Final test is prism cover test, the degree of eye misalignment is established by neutralizing the uncovered eye movements using a prism bar or prism lenses. This test should be performed separately for horizontal and vertical strabismus. However, this test requires a patient's cooperation and is quite complicated to perform. The cover and prism cover tests require the subjective judgment of the specialist to determine the existence of eye movements between consecutive cover/uncover steps. All the above tests are complicated to perform and are imprecise, prolonged, and have to be performed by a specialist.

In addition, virtual reality digital imaging equipment rise gradually in the market, but virtual reality head-mounted displays do not provide suitable images for people with poor vision. There is an urgent problem to solve in the market which is to provide suitable images on a head-mounted display device suitable for strabismus patients.

### SUMMARY

The present invention provides a head-mounted display device with built-in strabismus treatment. The head-mounted display device comprises a display unit, detecting unit, processing module, and graphic simulation unit. The display unit displays a test image to a user for test. The detecting unit detects a deviation angle of eyeballs of the user when the user focus on the test image. The processing module connects to the display unit and the detecting unit. The graphic simulation unit connects to the display unit and the processing module. The graphic simulation unit and the processing module obtain and analyze the deviation angle from the detecting unit, and the graphic simulation unit and the processing module output a compensated image to the display unit according to an analyzing result.

The operation method of the head-mounted display device of the embodiment of the present invention comprises follow steps: (A) providing a head-mounted display device of claim 1. (B) displaying the test image to the user for test. (C) detecting a deviation angle of eyeballs of the user when the user focus on the test image. (D) simulating to obtain the modified test image by the deviation angle from the detecting unit. (E) providing the modified test image to the user for test again and repeating step (B)-(E) until the deviation angle is equal to 0 degree and thereby the processing module obtain the compensated angle of the user. (F) the processing module generates the compensated image by the compensated angle, and the compensated image is displayed on the display unit for the user.

The above-mentioned descriptions are only preferred embodiments of the present invention and are not intended to limit the scope of implementation of the present invention. Therefore, all the shapes, structures, features, and spirits described in the scope of the patent application of the present invention shall be regarded as equivalent to the changes and modifications per se, and be included in the scope of the patent application of the present invention.

### BRIEF DESCRIPTION OF THE DRAWINGS

FIG. 1 is a schematic diagram of the head-mounted display device of the embodiment of the present invention.

FIG. 2 is a schematic diagram of tracking the eyeball movement of the user by the head-mounted display device of the embodiment of the present invention.

FIG. 3 is a flow chart of the operation method of the embodiment of the head-mounted display device.

### DETAILED DESCRIPTION OF THE INVENTION

Thereinafter, to make the description of the present disclosure more detailed and complete, the following description provides an illustrative description for the implementation and specific embodiments of the present invention. However, the following description is not the only form of implementing or using specific embodiments of the invention. In these paragraphs, the features of various specific embodiments are covered as well as the method steps and sequences for constructing and operating these specific embodiments. However, the other embodiments may also be utilized to achieve the same or equivalent function and sequence of steps.

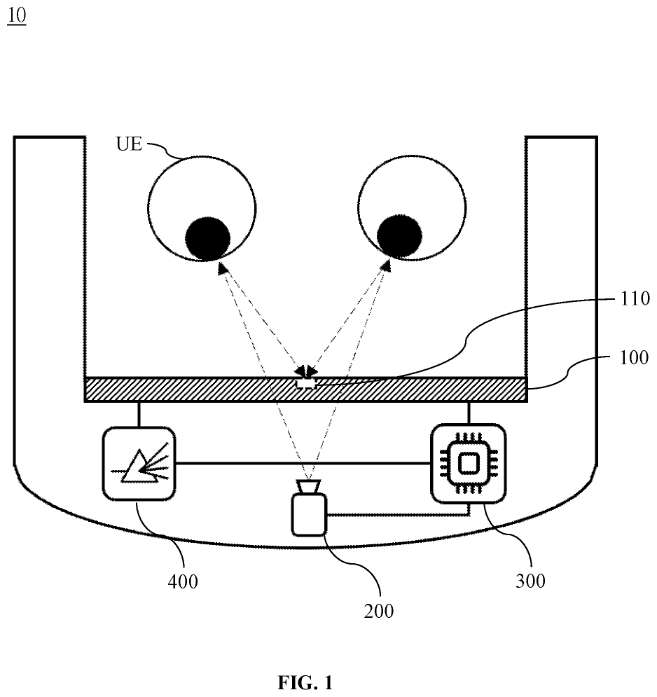

The present invention can inspect the defect of the eyes and generate compensated images to treat some people with visual disorders. FIG. 1 is a schematic diagram of the head-mounted display device of the embodiment of the present invention.

As shown in FIG. 1, the present invention provides a head-mounted display device 10 with built-in strabismus treatment. The head-mounted display device 10 comprises a display unit 100, a detecting unit 200, a processing module 300, and a graphic simulation unit 400. The display unit 100 displays a test image 110 to a user UE for test. The detecting unit 200 detects deviation angles of eyeballs of the user UE when the user UE focus on the test image 110. The processing module 300 connects to the display unit 100 and the detecting unit 200. The graphic simulation unit 400 connects to the display unit 100 and the processing module 300. The graphic simulation unit 400 and the processing module 300 obtain and analyze the deviation angle from the detecting unit 200, and the graphic simulation unit 400 and the processing module 300 output a compensated image to the display unit 100 according to the analyzing result.

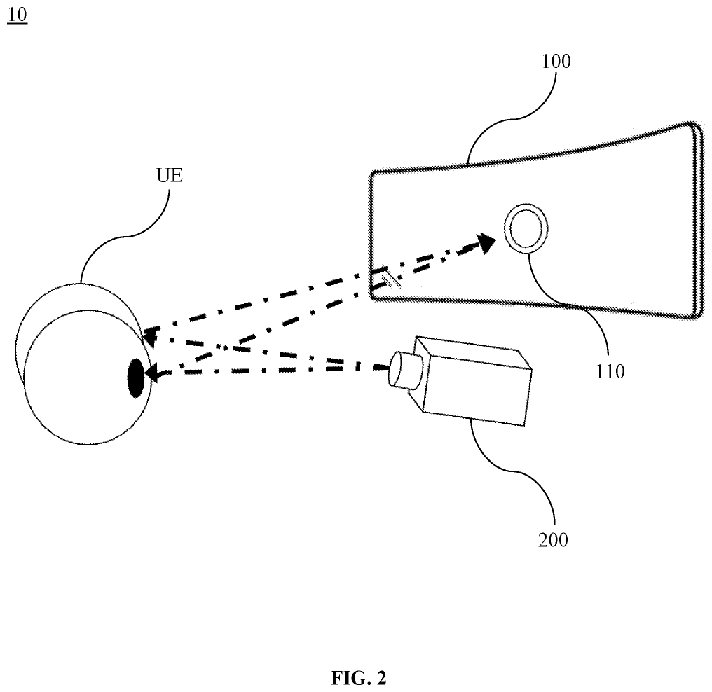

Furthermore, as shown in FIG. 2, the user UE puts on the head-mounted display device 10, and the user UE focus on the ring object of the test image 110. In the embodiment, the object is a ring. The object includes any shapes and color which is necessary to have clear features and simple shapes which easy to descript that make the user understand easily. As shown in FIG. 2, the detecting unit 200 further comprises an eye tracker which is a high-speed camera can catch the eye movements.

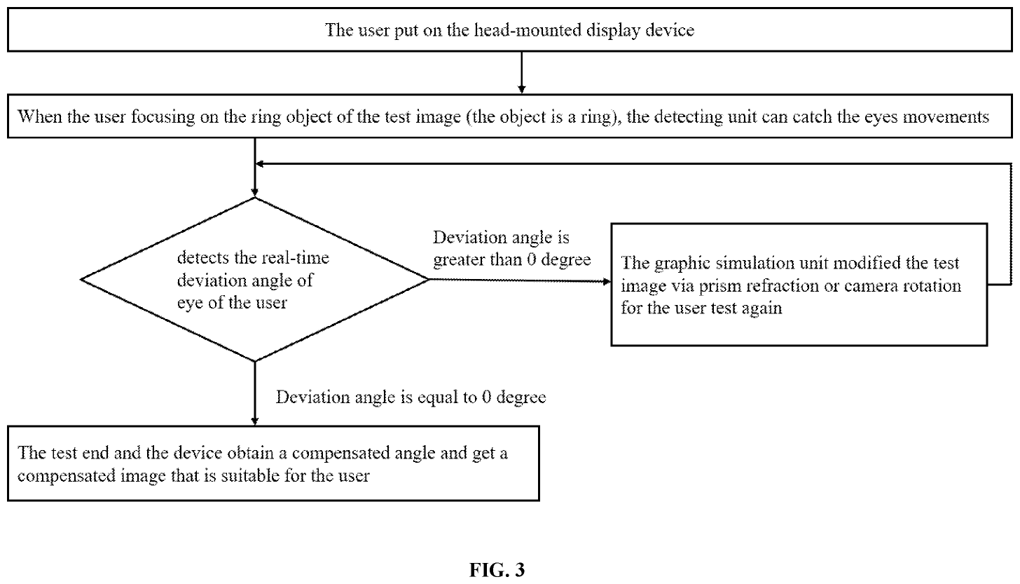

FIG. 3 is a schematic diagram of operation method of the head-mounted display device. As shown in FIG. 3, when the detecting unit 200 detects the deviation angle is equal to 0 degree, the test will end. If a person with normal ocular alignment will get the test result whose deviation angle is equal to 0 degree in the first test result. Conversely, the detecting unit 200 detects the deviation angle is greater than 0 degree, and the graphic simulation unit 400 modifies the test image 110 by the detecting deviation angle, wherein the simulated method includes prism simulating or camera rotation. The graphic simulation unit 400 outputs the modified test image to the display unit 100, and the user UE focus on the modified test image for test again. The test will end until the deviation angle is equal to 0 degree, and then the graphic simulation unit 400 and the processing module 300 will obtain a compensated angle.

In the final step of the test, when the device is detecting the user's the deviation angle is equal to 0 degree, the processing module 300 will get the compensated angle. The processing module 300 comprises the central processing unit and graphic processing unit. The central processing unit cooperates with the graphic processing unit to process by the compensated angle, and get a compensated image that is suitable for the user UE (i.e. strabismus treatment image). The compensated images of each eye display respectively on the head-mounted display device 10 make the user UE get the best visual experience. In addition, the head-mounted display device 10 will modify the rotation angle, and the deflection angle of the image to integrate the simulated result from prism diopter lens simulation system. Hence, the above simulated result makes the head-mounted display device display these images which are as same as prism diopter lens simulation system generated. Moreover, when the virtual reality function of the head-mounted display device 10 turns on, the processing unit 300 will output the compensated image which decreases the compensated angle. The decreased angle is calculated from the head-mounted display device 10 detects automatically the eye real-time movement of the user UE. The decreased angle is advantage to improve the user's strabismus and the head-mounted display device 10 could achieve visual treatment.

Specifically, during the user UE uses the application or plays virtual reality games, if the detecting unit 200 detects a significant movement of the user's eyes or head, the processing unit 300 will adjust the prism diopter to simulate the lens with a lower resolution. The prism diopter is defined as when the patient's left eye focus on an object placed 6 meters in front of the eyes (the middle of the field of vision), the place where the right eye focus on will deflect a distance relative to the middle.

According to the research, when the movement of user's head or eye is more than a certain range, the user cannot find the slight change of the scene. Hence, this adjustment of the prism diopter will not be felt easily by the user. Furthermore, the user will adapt to the environment with lower degree after using for long-term, and that can achieve the vision treatment.

The above-mentioned descriptions are only preferred embodiments of the present invention and are not intended to limit the scope of implementation of the present invention. Therefore, all the shapes, structures, features, and spirits described in the scope of the patent application of the present invention shall be regarded as equivalent to the changes and modifications per se, and be included in the scope of the patent application of the present invention.

What is claimed is:

1. A virtual reality head-mounted display device with built-in strabismus treatment, comprising:
a display unit displaying a test image to a user for test;
a detecting unit detecting a deviation angle of eyeballs of the user when the user focus on the test image;
a processing module, connected to the display unit and the detecting unit; and
a graphic simulation unit, connected to the display unit and the processing module;
wherein the graphic simulation unit and the processing module obtain and analyze the deviation angle from the detecting unit, and the graphic simulation unit and the processing module output a compensated image to the display unit according to an analyzing result.

2. The head-mounted display device as claimed in claim 1, wherein the test image comprises an object.

3. The head-mounted display device as claimed in claim 1, wherein if the detecting unit detects the deviation angle which is greater than 0 degree, the graphic simulation unit will simulate by the deviation angle, and then the graphic simulation unit output at least one modified test image to the display unit, the user focus on the modified test image for test;
wherein the test will end until the deviation angle is equal to 0 degree, and then the graphic simulation unit and the processing module will obtain a compensated angle.

4. The head-mounted display device as claimed in claim 3, wherein the processing module processes an optical simulation by the compensated angle, and the processing module generate the compensated image; the compensated image is displayed on the display unit for the user.

5. The head-mounted display device as claimed in claim 1, wherein the graphic simulation unit modified the test image via prism refraction or camera rotation.

6. The head-mounted display device as claimed in claim 1, wherein the processing module is a central processing unit, graphic processing unit, or combinations thereof.

7. The head-mounted display device as claimed in claim 1, wherein the display unit is activated actively or passively.

8. The head-mounted display device as claimed in claim 1, wherein the display unit comprises a 3D display device.

9. The head-mounted display device as claimed in claim 1, wherein the detecting unit comprises a camera, and the detecting unit further comprises an eye tracker.

10. An operation method of a head-mounted display device with built-in strabismus treatment, comprises:
(A) providing a head-mounted display device of claim 1;
(B) displaying the test image to the user for test;
(C) detecting a deviation angle of eyeballs of the user when the user focus on the test image;
(D) simulating to obtain the modified test image by the deviation angle from the detecting unit;
(E) providing the modified test image to the user for test again and repeating step (B)-(E) until the deviation angle is equal to 0 degree and thereby the processing module obtain the compensated angle of the user; and
(F) the processing module generates the compensated image by the compensated angle, and the compensated image is displayed on the display unit for the user.
